(19) United States
(12) Patent Application Publication
(10) Pub. No.: US 2023/0398087 A1
(43) Pub. Date: Dec. 14, 2023

(54) APPLICATION OF BZP IN TREATMENT OF ISCHEMIC CARDIOVASCULAR AND CEREBRAL VASCULAR DISEASES

(71) Applicant: ZHEJIANG AUSUN PHARMACEUTICAL CO., LTD., Linhai (CN)

(72) Inventor: Zhiguo ZHENG, Linhai (CN)

(73) Assignee: ZHEJIANG AUSUN PHARMACEUTICAL CO., LTD., Linhai (CN)

(21) Appl. No.: 18/034,144

(22) PCT Filed: Oct. 28, 2021
(86) PCT No.: PCT/CN2021/127101

§ 371 Date: Apr. 27, 2023

(30) Foreign Application Priority Data
Oct. 30, 2020 (CN) 202011195601.4

(51) Int. Cl. A61K 31/192; A61P 9/10

(52) U.S. Cl. CPC A61K 31/192; A61P 9/10

(57) ABSTRACT

The present invention relates to an application of 5-bromo-2-(α-hydroxyamyl) sodium benzoate salt (BZP) in treatment of ischemic cardiovascular and cerebral vascular diseases, in particular to a BZP compound for treatment of ischemic stroke and a treatment method, and more specifically, relates to a treatment solution for treating mild and moderate acute ischemic stroke by using the BZP and a pharmaceutical composition comprising the BZP.

### TECHNICAL FIELD

The invention relates to the field of organic chemical drug therapy. In particular, the present invention relates to compounds and methods for the treatment of ischemic stroke, more specifically, the present invention relates to a treatment regimen for treatment of mild or moderate acute ischemic stroke by using sodium 5-bromo-2-(α-hydroxypentyl)benzoate (BZP), and a pharmaceutical composition comprising the BZP.

### BACKGROUND ART

Acute stroke is a disease with high morbidity, high disability, high mortality, high recurrence and many complications, which seriously endangers human health. Survivors often leave sequalae, which affect the quality of life of patients and increase the burden on their families and society. At present, the effective treatment for acute ischemic stroke (AIS) is to give vascular recanalization therapy including recombinant tissue plasminogen activator for intravenous thrombolysis and mechanical thrombectomy within a certain time window. Although it has good results, it is far from meeting the clinical needs. Therefore, finding effective drugs for treating acute ischemic stroke has always been a problem to be solved in this field.

Chinese patent application CN101402565A disclosed for the first time halogen-substituted 2-(α-hydroxypentyl) benzoate compounds and their preparation methods, and their use in the prevention and treatment of cardio-cerebral ischemic diseases, improvement of cardio-cerebral circulation disorders, anti-thrombosis and the like. Wherein, it mentions the preparation of salt of monovalent metal ion, divalent metal ion, trivalent metal ion or organic base that is soluble in water, and specifically mentions potassium salt, calcium salt and phenethylamine salt etc.

PCT patent application WO2015007181A disclosed sodium 5-bromo-2-(α-hydroxypentyl)benzoate (BZP) compound and amorphous form, crystalline form A and crystalline form B and preparation methods thereof, as well as their use in the manufacture of a medicament for prevention and treatment of cardiovascular and cerebrovascular diseases, improving cardio-cerebral circulation disorders or anti-thrombosis.

However, the above-mentioned patent applications have only verified the activity in animals in terms of use of drugs for the prevention and treatment of cardio-cerebral ischemic diseases, the improvement of cardio-cerebral circulation disorders, and anti-thrombosis. Considering the complexity and particularity of human-related diseases, it is still necessary to conduct in-depth research and improvement on the administration method and dosage regimen for the use of such drugs.

### DISCLOSURE OF THE INVENTION

### 1. Treatment Method

In a first aspect, the present invention relates to a method for preventing or treating cardio-cerebral ischemic diseases or improving cardio-cerebral circulation disorder or anti-thrombosis in an individual, the method comprising administering an effective amount of sodium 5-bromo-2-(α-hydroxypentyl)benzoate (BZP) compound to the individual.

The BZP compound has the following structure:

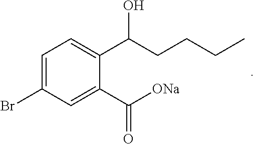

In one embodiment, the BZP compound is in the form of an amorphous, crystalline form A or crystalline form B, and their characteristics are as described in WO2015007181A, the entire content of which is incorporated herein by reference.

In one embodiment, the disease is acute stroke and related diseases. In a preferred embodiment, the disease is mild or moderate acute ischemic stroke.

In one embodiment, the disease is one characterized by:
(1) Acute ischemic stroke;
(2) Acute ischemic stroke, cerebral anterior circulation infarction;
(3) Acute ischemic stroke, cerebral anterior circulation infarction, non-cardiogenic infarction;
(4) Acute ischemic stroke, cerebral anterior circulation infarction, non-cardiogenic infarction, moderate severity (NIHSS 5-15 scores)
(5) Acute ischemic stroke, cerebral anterior circulation infarction, non-cardiogenic infarction, moderate severity (NIHSS 5-15 scores), aged 45-85 years; or
(6) Acute ischemic stroke, anterior cerebral circulation infarction, non-cardiogenic infarction, moderate severity (NIHSS 5-15 scores), aged 45-85 years old, BZP needs to be administered within 24 hours of stroke onset.

In one embodiment, the individual is a human individual.

In a preferred embodiment, the individual is a human individual having the diseases, conditions and/or characteristics of any one of the above embodiments.

In a preferred embodiment, the individual suffers from a specific disease characterized by:
(1) Acute ischemic stroke;
(2) Acute ischemic stroke, cerebral anterior circulation infarction;
(3) Acute ischemic stroke, cerebral anterior circulation infarction, non-cardiogenic infarction;
(4) Acute ischemic stroke, cerebral anterior circulation infarction, non-cardiogenic infarction, moderate severity (NIHSS 5-15 scores);
(5) Acute ischemic stroke, cerebral anterior circulation infarction, non-cardiogenic infarction, moderate severity (NIHSS 5-15 scores), aged 45-85 years; or
(6) Acute ischemic stroke, anterior cerebral circulation infarction, non-cardiogenic infarction, moderate severity (NIHSS 5-15 scores), aged 45-85 years, BZP needs to be administered within 24 hours of stroke onset.

In a preferred embodiment, the time from stroke onset to administration in said individual is no more than 24 hours. In a more preferred embodiment, the individual is a human patient with a time from stroke onset to administration of 6-24 hours.

In a preferred embodiment, the treatment results in one or more of the following improvements in the individual:
(1) Improving the neurological function score and reducing the NIHSS score;
(2) Improving neurological function outcomes and reducing mRS scores;
(3) Reducing the volume of cerebral infarction;
(4) Reducing the recurrence of ischemic stroke;
(5) Reducing the hemorrhagic transformation of ischemic stroke, including symptomatic intracranial hemorrhage and asymptomatic hemorrhagic transformation; and/or
(6) Reducing the mortality of ischemic stroke.

In some embodiments, the BZP according to the invention are formulated for administration as a liquid pharmaceutical composition. The suitable vehicles and solvents include water, Ringer's solution, phosphate-buffered saline, isotonic sodium chloride solution, and the like. In addition, sterile, fixed oils may be employed as a solvent or suspending medium, where appropriate. For this purpose, any mixture of fixed mineral or non-mineral oils, including synthetic mono- or diglycerides, may be employed. In addition, fatty acids such as oleic acid can also be used for preparing injections.

In some embodiments, the pharmaceutical composition comprising the BZP according to the invention is a solution or dry powder for injection. For example, the composition is a lyophilized powder, which can be reconstituted as an injection in a pharmaceutically acceptable liquid carrier. The pharmaceutically acceptable liquid carrier can be, for example, sterile water, Ringer's solution, phosphate-buffered saline, isotonic sodium chloride solution and the like.

In some embodiments, the BZP according to the invention are administered parenterally.

In some embodiments, the BZP according to the invention are administered by intravenous injection.

In some embodiments, the BZP according to the invention are administered by intravenous infusion.

In some embodiments, the daily dose of the BZP according to invention is 50-500 mg, such as 100-500 mg, 200-500 mg, 250-500 mg, 300-500 mg, 325-475 mg, 350-475 mg, 350-450 mg, 375-450 mg, or 375-425 mg, for example 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg or 500 mg.

In some embodiments, the single dose of the BZP according to the invention is 25-400 mg, such as 50-400 mg, preferably 50-300 mg, more preferably 50-250 mg, even more preferably 100-250 mg, such as 25 mg, 50 mg, 75 mg, 100 mg, 125 mg, 150 mg, 175 mg, 200 mg, 225 mg, 250 mg, 275 mg, 300 mg, 325 mg, 350 mg, 375 mg or 400 mg; the BZP according to the invention is administered three times a day, twice a day, once a day, once every two days, once every three days, twice a week, once a week, once every two weeks, once every four weeks or longer. The administration cycle of the BZP according to the invention can be one week, two weeks, three weeks, one month, two months, three months or longer, and the intervals between each administration cycle can be the same or different.

In a preferred embodiment, the dosage regimen of the BZP according to invention is once to three times a day, with a dose of 50-250 mg each time, for example, once, twice or three times a day, with a dose of 50 mg, 75 mg, 100 mg, 125 mg, 150 mg, 175 mg, 200 mg, 225 mg or 250 mg each time. Preferably, the dosage regimen is twice a day, with a dose of 100-250 mg each time, for example, twice a day, with a dose of 100 mg, 125 mg, 150 mg, 175 mg, 200 mg, 225 mg or 250 mg each time.

In some embodiments, the BZP according to the invention or its pharmaceutical composition can be administered alone or in combination with other drugs.

In some embodiments of the above methods, the subject is free of serious adverse events following administration.

In some embodiments of the above methods, the subject has an incidence of adverse events comparable to a subject receiving placebo following administration.

In a preferred embodiment, the individual has a lower incidence of adverse events under the following dosage regimen than other dosage regimens:

The BZP compound is administered one to three times a day, with a dose of 100-250 mg each time, for example it is administered twice a day, with a dose of 100 mg, 125 mg, 150 mg, 175 mg, 200 mg, 225 mg or 250 mg each time, preferably it is administered twice a day, with a dose of 100 mg, 125 mg, 150 mg, 175 mg, 200 mg or 225 mg each time, more preferably it is administered twice a day, with a dose of 200 mg each time.

In a more preferred embodiment, the individual has a lower incidence of adverse events under the following dosage regimen than other dosage regimens and achieves better improved effects than other dosage regimens:

The BZP compound is administered one to three times a day, with a dose of 100-250 mg each time, for example it is administered twice a day, with a dose of 100 mg, 125 mg, 150 mg, 175 mg, 200 mg, 225 mg or 250 mg each time, preferably it is administered twice a day, with a dose of 100 mg, 125 mg, 150 mg, 175 mg, 200 mg or 225 mg each time, more preferably it is administered twice a day, with a dose of 200 mg each time.

In some embodiments of the above methods, the individual is a human. In a preferred embodiment, the time from stroke onset to administration in said individual is no more than 24 hours. In a more preferred embodiment, the individual is a human patient with a time from stroke onset to administration of 6-24 hours.

### 2. Use

In a second aspect, the present invention relates to the use of BZP in the manufacture of a medicament for preventing or treating cardio-cerebral ischemic diseases or improving cardio-cerebral circulation disorders or anti-thrombosis in individuals.

In a third aspect, the present invention relates to BZP, for use in preventing or treating cardio-cerebral ischemic diseases or improving cardio-cerebral circulation disorders or anti-thrombosis in individuals.

For the second and third aspects, they are further defined as follows:

In one embodiment, the specified disease is characterized by:
(1) Acute ischemic stroke;
(2) Acute ischemic stroke, cerebral anterior circulation infarction;
(3) Acute ischemic stroke, cerebral anterior circulation infarction, non-cardiogenic infarction;
(4) Acute ischemic stroke, cerebral anterior circulation infarction, non-cardiogenic infarction, moderate severity (NIHSS 5-15 scores)
(5) Acute ischemic stroke, cerebral anterior circulation infarction, non-cardiogenic infarction, moderate severity (NIHSS 5-15 scores), aged 45-85 years; or
(6) Acute ischemic stroke, anterior cerebral circulation infarction, non-cardiogenic infarction, moderate severity (NIHSS 5-15 scores), aged 45-85 years, BZP needs to be administered within 24 hours of stroke onset.

In one embodiment, the individual is a human individual.

In a preferred embodiment, the individual is a human individual having the diseases, conditions and/or characteristics of any one of the above embodiments.

In a preferred embodiment, the individual is a human individual, who is a patient suffering from a specific disease with geographic atrophy.

In a preferred embodiment, the individual suffers from a specific disease characterized by:
(1) Acute ischemic stroke;
(2) Acute ischemic stroke, cerebral anterior circulation infarction;
(3) Acute ischemic stroke, cerebral anterior circulation infarction, non-cardiogenic infarction;
(4) Acute ischemic stroke, cerebral anterior circulation infarction, non-cardiogenic infarction, moderate severity (NIHSS 5-15 scores)
(5) Acute ischemic stroke, cerebral anterior circulation infarction, non-cardiogenic infarction, moderate severity (NIHSS 5-15 scores), aged 45-85 years; or
(6) Acute ischemic stroke, anterior cerebral circulation infarction, non-cardiogenic infarction, moderate severity (NIHSS 5-15 scores), aged 45-85 years, BZP needs to be administered within 24 hours of stroke onset.

In a preferred embodiment, the treatment achieves one or more of the following effects in the individual:
(1) Improving the neurological function score and reducing the NIHSS score;
(2) Improving neurological function outcomes and reducing mRS scores;
(3) Reducing the volume of cerebral infarction;
(4) Reducing the recurrence of ischemic stroke;
(5) Reducing the hemorrhagic transformation of ischemic stroke, including symptomatic intracranial hemorrhage and asymptomatic hemorrhagic transformation; and/or
(6) Reducing the mortality of ischemic stroke.

In some embodiments, the daily dose of the BZP according to invention is 50-500 mg, such as 100-500 mg, 200-500 mg, 250-500 mg, 300-500 mg, 325-475 mg, 350-475 mg, 350-450 mg, 375-450 mg, or 375-425 mg, for example 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg or 500 mg.

In some embodiments, the single dose of the BZP according to the invention is 25-400 mg, such as 50-400 mg, preferably 50-300 mg, more preferably 50-250 mg, even more preferably 100-250 mg, such as 25 mg, 50 mg, 75 mg, 100 mg, 125 mg, 150 mg, 175 mg, 200 mg, 225 mg, 250 mg, 275 mg, 300 mg, 325 mg, 350 mg, 375 mg or 400 mg; the BZP according to the invention is administered three times a day, twice a day, once a day, once every two days, once every three days, twice a week, once a week, once every two weeks, once every four weeks or longer. The administration cycle of the BZP according to the invention can be one week, two weeks, three weeks, one month, two months, three months or longer, and the intervals between each administration cycle can be the same or different.

In a preferred embodiment, the dosage regimen of the BZP according to invention is once to three times a day, with a dose of 50-250 mg each time, for example, once, twice or three times a day, with a dose of 50 mg, 75 mg, 100 mg, 125 mg, 150 mg, 175 mg, 200 mg, 225 mg or 250 mg each time. Preferably, the dosage regimen is twice a day, with a dose of 100-250 mg each time, for example, twice a day, with a dose of 100 mg, 125 mg, 150 mg, 175 mg, 200 mg, 225 mg or 250 mg each time.

In some embodiments, the BZP according to invention or a pharmaceutical composition thereof can be administered alone or in combination with other drugs. The other drugs used in combination include, but are not limited to, edaravone, citicoline, piracetam (naofukang), butylphthalide, and human urinary kallidinogenase.

In some embodiments, the subject is free of serious adverse events following administration.

In some embodiments, the subject has an incidence of adverse events comparable to subjects receiving placebo following administration.

In some embodiments, the individual is a human.

It should be understood that the technical solution obtained by any combination of any technical features described in the second to third aspects above and any technical features of the technical solution described in the first aspect is also included in this application.

### 3. Single Dosage Units or Kits

In a fourth aspect, the present invention relates to a single dosage unit characterized in that it comprises a BZP compound.

The BZP compound has the following structure:

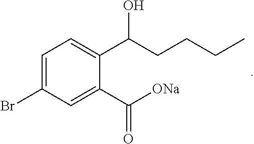

In one embodiment, the BZP compound is in the form of an amorphous, crystalline form A or crystalline form B, and their characteristics are as described in WO2015007181A, the entire content of which is incorporated herein by reference.

In a preferred embodiment, the single dosage unit comprises the BZP compound in the dose of 25-400 mg, such as 50-400 mg, preferably 50-300 mg, more preferably 50-250 mg, even more preferably 100-250 mg, for example 25 mg, 50 mg, 75 mg, 100 mg, 125 mg, 150 mg, 175 mg, 200 mg, 225 mg, 250 mg, 275 mg, 300 mg, 325 mg, 350 mg, 375 mg or 400 mg.

In a further preferred embodiment, the single dosage unit may be a lyophilized powder or a liquid preparation. The “liquid preparation” or “liquid composition” refers to a preparation in liquid form. The liquid composition according to the invention comprises (i) the BZP compound according to the invention; and (iii) a pharmaceutically acceptable liquid carrier. The liquid preparation according to the invention is preferably an injection, such as a liquid preparation via intravenous injection or infusion. Alternatively, the composition according to the invention is a lyophilized powder formulation, which can be reconstituted as an injection in a pharmaceutically acceptable liquid carrier. The pharmaceutically acceptable liquid carrier can be, for example, sterile water, Ringer's solution, phosphate-buffered saline, isotonic sodium chloride solution and the like.

In a fifth aspect, the present invention relates to a kit, characterized in that it comprises one or more single dosage units according to any one of the embodiments of the above single dosage unit. In a preferred embodiment, the kit further comprises a package insert for instructing the use of the single dosage unit.

It should be understood that the technical solution obtained by any combination of any technical features described in the fourth to fifth aspects above and any technical features of the technical solution described in the first to third aspects is also included in this application.

### Definition

In order to explain this specification, the following definitions will be used, and whenever appropriate, terms used in the singular may also include the plural and vice versa. It is to be understood that the terminology used herein is for the purpose of describing particular embodiments only and is not intended to be limiting.

As used herein, the term “comprising” or “including” means including stated elements, integers or steps, but not excluding any other elements, integers or steps. Herein, when the term “comprising” or “including” is used, unless otherwise specified, it also covers the situation of combining the mentioned elements, integers or steps. For example, when referring to an antibody variable region that “comprises” a specific sequence, it is also intended to encompass an antibody variable region that consists of that specific sequence.

“Individual” includes mammals. Mammals include, but are not limited to, domesticated animals (e.g., cattle, sheep, cats, dogs, and horses), primates (e.g., humans and non-human primates such as monkeys), rabbits, and rodents (e.g., mice and rats). In some embodiments, the individual is a human, including a child, adolescent, or adult.

As used herein, “treatment” means slowing, interrupting, arresting, alleviating, stopping, reducing, or reversing the progression or severity of an existing symptom, disorder, condition, or disease.

As used herein, “prevention” includes the inhibition of the occurrence or development of a disease or disorder or a symptom of a particular disease or disorder. In some embodiments, individuals with a family history of the disease are candidates for preventing regimens. In general, the term “prevention” refers to the administration of a drug prior to the onset of signs or symptoms, especially in at-risk individuals.

The term “effective amount” refers to an amount effective, at dosages required, and for periods of time required, to achieve the desired therapeutic result. A therapeutically effective amount of a preparation, BZP compound or composition according to the invention may vary depending on factors such as disease state, age, sex and weight of the individual and the ability of the antibody or antibody portion to elicit a desired response in the individual. A therapeutically effective amount is also one in which any toxic or detrimental effects of the preparation, BZP compound or composition are lower than the therapeutically beneficial effects.

A “preventively effective amount” refers to an amount effective, at dosages required, and for periods of time required, to achieve the desired preventive result. Typically, a preventively effective amount will be less than a therapeutically effective amount because the preventive dosage is administered in the subject before or at an earlier stage of the disease.

As used herein, the term “preparation” or “pharmaceutical composition” refers to a composition comprising at least one active ingredient and at least one inactive ingredient suitable for administration to an animal, preferably a mammal, including a human. The preparation according to the invention may be a lyophilized powder or a liquid preparation. “Liquid preparation” or “liquid composition” refers to a preparation in liquid form. The liquid composition according to the invention comprises (i) the BZP compound according to the invention; and (iii) a pharmaceutically acceptable liquid carrier. The liquid preparation according to the invention is preferably an injection, such as a liquid preparation via intravenous injection or infusion, such as a liquid preparation prepared in 0.9% sodium chloride solution.

“Pharmaceutically acceptable carrier” refers to ingredients in pharmaceutical preparations other than the active ingredient, which are nontoxic to the subject. Pharmaceutically acceptable carriers include, but are not limited to, solvents, buffers, excipients, stabilizers or preservatives.

The term “about” when used in conjunction with a numerical value is meant to encompass a numerical value within a range having a lower limit 10% less than the stated numerical value and an upper limit 10% greater than the stated numerical value.

As used herein, the term “single dosage unit” refers to a single dosage form comprising a BZP compound according to the invention to be administered to a patient, such as a vial for injection, an ampoule, an infusion bag, a prefilled needle or a prefilled syringe, a solution or lyophilized powder that contains the drug.

### EXAMPLES

The present invention is further described below in conjunction with the examples, and the following examples should not be construed as limiting the scope of the present invention.

### Abbreviation

AE adverse event
DLT dose limiting toxicity
EC50 half effective concentration
SAE serious adverse event
TEAE treatment emerged adverse event

### Example 1: Pharmacological Studies in Animals

Compared with the model group, BZP at doses of 3 mg/kg, 12 mg/kg and 24 mg/kg can significantly reduce the volume of cerebral infarction and brain swelling in MCAO model rats, and improve the neurobehavior of MCAO model rats and salt-sensitive rats in a dose-dependent manner. At a dose of 12 mg/kg, BZP can significantly restore muscle strength and balance beam behavior in MCAO model rats, and reduce the proportion of necrotic neurons and apoptosis rate, and the improved effects are significantly better than those of NBPK group. In addition, BZP 12 mg/kg can reduce the mortality and behavioral score within 24 hours of the global cerebral ischemia-reperfusion model in rats, and significantly reduce the blood pressure of salt-sensitive rats 21 days after administration. BZP has a protective effect on cerebral ischemia-reperfusion injury and is effective when administered within 4 hours after ischemia, especially within 2 hours after ischemia.

In terms of the respiratory system, a single intravenous injection of BZP 25-100 mg/kg had no effect on rats.

In terms of neurobehavior, a single intravenous injection of BZP 50 mg/kg and 100 mg/kg can cause a decrease in body temperature in rats; rats at a high dose (100 mg/kg) also have decreased activity, erect hair, decreased frequency of defecation and urination and loss of pupillary stimulation reflex, etc. These changes all recovered within 4 hours after drug withdrawal. The effective dose (12 mg/kg) of BZP to protect against ischemia-reperfusion injury in rats was lower than the dose (50 mg/kg) that caused neurological effects in rats.

In terms of the cardiovascular system, there was a small transient increase in systolic blood pressure, diastolic blood pressure, and mean arterial pressure in dogs 0.25 hours after intravenous injection of 50 mg/kg BZP, which has a certain dose correlation. These changes all recovered within 0.5 hours after administration. The dose range of 5-50 mg BZP has no effect on the II-lead ECG.

### Example 2: Pharmacodynamics Study in Animals

2.1 The Protective Effect of BZP on Focal Cerebral Ischemia-Reperfusion Injury in Rats:
(1) Changes in neurobehavioral scores: compared with the model group, the 3, 12 and 24 mg/kg of BZP groups had significantly lower scores at 2.5 h and 6 h (P<0.05, P<0.01), in a dose-dependent manner, indicating that BZP can significantly improve the motor dysfunction in rats with cerebral ischemia-reperfusion injury;
(2) Changes in the volume of cerebral infarction: compared with the model group, the volumes of cerebral infarction in 0.75, 3, 12 and 24 mg/kg of BZP groups were significantly reduced (P<0.01) in a dose-dependent manner, and the effects of 12 and 24 mg/kg of BZP groups were better than those of NBP-K and Br-NBP groups (P<0.01), and the effect of 3 mg/kg of BZP group was better than that of Br-NBP group (P<0.01). It shows that BZP can significantly reduce the volume of cerebral infarction. 0.75, 3 and 12 mg/kg of BZP groups were analyzed for the volumes of cortical and striatal infarction, 0.75 mg/kg of BZP group can significantly reduce the volume of cortex (P<0.01), it was found that 3 and 12 mg/kg of BZP groups significantly reduced the volumes of striatal and cortical infarction (P<0.01).
(3) Changes in brain edema: compared with the model group, the volumes of brain edema in the 3, 12 and 24 mg/kg of BZP groups were significantly reduced (P<0.01), in a dose-dependent manner.

The effects of 24 and 12 mg/kg of BZP groups were better than those of NBP-K group and Br-NBP group (P<0.01);
(4) Treatment time window: compared with the model group, administration of 12 mg/kg of BZP at 0.5 h, 2 h or 4 h after ischemia could significantly reduce the volume of cerebral infarction (P<0.01, P<0.05). The effects of 0.5 h and 2 h was better than that of 4 h (P<0.01). In the follow-up test, the drug was administered at 2 h of ischemia.

BZP can improve motor dysfunction, reduce the volume of cerebral infarction, and reduce cerebral edema. The optimal treatment time window is within 4 hours after ischemia, especially within 2 hours after ischemia.

2.2 Effects of BZP on Inflammatory Response Induced by Focal Cerebral Ischemia-Reperfusion Injury in Rats:

The results of TNF-α, IL-6, IL-1β, NF-κB, Cox-2, ICAM-1 immunohistochemical staining

The results of immunohistochemical staining optical density value showed: compared with the model group, the expression of TNF-α, IL-6, IL-1β, NF-κB, COX-2, ICAM-1 in low, middle and high dose of BZP groups were significantly reduced (P<0.05, P<0.01), and 12 mg/kg of BZP group was better than 9.6 mg/kg of NBP-K group and 10.5 mg/kg of Br-NBP group (P<0.05, P<0.01).

The results of the number of immunohistochemical positive cells showed: compared with the model group, the expressions of TNF-α, IL-6, IL-1β, NF-κB, COX-2, and ICAM-1 in low, middle and high dose of BZP groups were significantly reduced (P<0.05, P<0.01), and 12 mg/kg of BZP group was better than 9.6 mg/kg of NBP-K group and 10.5 mg/kg of Br-NBP group (P<0.05, P<0.01).

BZP can inhibit the cellular inflammatory response induced by cerebral ischemia-reperfusion injury, manifested as inhibiting the expression of TNF-α-IL-6-IL-1β-NF-κB-COX-2-ICAM-1 loop.

2.3 The Protective Effect of BZP on Neuronal Apoptosis after Focal Cerebral Ischemia-Reperfusion Injury in Rats:

BZP can reduce the loss of nerve cells and reduce the apoptosis rate, which is manifested by inhibiting the Bax/Bcl-2-cytochrome C-Caspase-3 mitochondrial apoptosis pathway and up-regulating the PI3K-p-Akt survival signaling pathway, that is, BZP has the effect of inhibiting apoptosis.

2.4 Effect of BZP on Neuroplasticity after Focal Cerebral Ischemia-Reperfusion Injury in Rats:

BZP can promote nerve repair after cerebral ischemic injury, improve the recovery of motor function after cerebral ischemic injury in rats, and its mechanism of action is related to increasing the expression of SYN, GAP-43, BDNF, and GFAP, and down-regulating NOGO-A expression, in the ischemic penumbra area.

2.5 The Protective Effect of BZP on Global Cerebral Ischemia-Reperfusion Injury in Rats:

The mortalities within 24 hours in the BZP (3 and 12 mg/kg) groups was lower than those in the model group and the NBP-K group; the behavioral scores in the BZP groups were significantly lower than that in the model group (P<0.05); compared with the model group, the expression of inflammatory circuit factors (TNF-α, IL-1β, IL-6, NF-κB, COX-2, ICAM-1), apoptosis pathway factors (Bax/Bcl-2, Cyt-c, Caspase-3), neuron necrosis rate and apoptosis rate in hippocampal CA1 region in BZP group were significantly decreased (P<0.05), and cell survival pathways (PI3K, P-Akt) were significantly increased (P<0.01).

BZP has a protective effect on acute global cerebral ischemia in rats, and its mechanism is to inhibit the inflammatory response and apoptosis of cells.

2.6 Anti-Platelet Aggregation and Anti-Thrombosis Effects of BZP:

Ex vivo tests showed that BZP exhibited a dose-dependent inhibitory effect on AA-induced platelet aggregation, with an IC₅₀ of 12 μmol/L, but had no inhibitory effects on ADP and thrombin-induced platelet aggregation; rat in vivo tests showed that each dose of BZP groups had the effects against platelet aggregation induced by ADP (platelet aggregation inhibition rates were 23.89%, 34.42% and 44.14%, respectively), thrombin (platelet aggregation inhibition rates were 23.87%, 37.75% and 55.47%, respectively), arachidonic acid (platelet aggregation inhibition rates were 42.08%, 60.32% and 72.20%, respectively) in a concentration-dependent manner; experimental thrombotic changes: the results of experimental thrombosis in low, medium and high dose of BZP groups were 0.23±0.04, 0.18±0.02, 0.13±0.03, which was significantly lower (P<0.01) compared with the model group, and it was in a dose-dependent manner. Among them, BZP in the high dose group had no statistical difference compared with the ASA group with the same molar dose (P>0.05), but was weaker than that in the Br-NBP group (P<0.01). The results showed that BZP had an effect of anti-thrombosis.

Ex vivo tests showed that BZP could selectively inhibit platelet aggregation induced by AA; in vivo tests showed that BZP could inhibit platelet aggregation induced by ADP, thrombin and AA; BZP could inhibit the experimental thrombosis in rats.

2.7 The Protective Effect of BZP on Acute Global Cerebral Ischemia in Rats:

Compared with the sham operation group, the rats in the model group had a larger volume of cerebral infarction, while 0.75, 3, 12 and 24 mg/kg of BZP groups could significantly reduce the volume of cerebral infarction (P<0.05, P<0.01); 10.5 mg/kg of Br-NBP group and 9.6 mg/kg of NBP-K group could also significantly reduce the volume of cerebral infarction (P<0.01), but the reductions of the volume of cerebral infarction in 12 and 24 mg/kg of BZP groups were better than those in Br-NBP group and NBP-K group (P<0.01). The improvement of BZP on the volume of cerebral infarction in rats showed an obvious dose-effect relationship. With the increase of dose, the volume of cerebral infarction in rats gradually decreased, which had a high correlation (R²=0.9739).

The results of nuclear magnetic resonance showed that: BZP has a protective effect on focal cerebral ischemia-reperfusion injury in rats.

### Example 3: Pharmacokinetic Study in Animals

After a single intravenous injection of BZP in rats (0.75-12.0 mg/kg) and dogs (1.0-4.0 mg/kg), the Cₘₐₓ and AUC₀₋ₜ of BZP and BNBP both increased with increasing doses, which basically conformed to linear kinetics. Except that the AUC₀₋ₜ of BZP in female rats was significantly higher than that in male rats, there was no significant difference between male and female for BZP and BNBP in dogs and BNBP in rats.

When BZP was injected intravenously for 7 consecutive days in rats (6.0 mg/kg) and dogs (2.0 mg/kg), BZP and BNBP did not accumulate in the body. In rats, the AUC₀₋ₜ of BZP after the last administration was significantly higher than that of the first time, but there was no difference in Cₘₐₓ between a single dose and multiple doses, indicating that there may be changes in BZP and BNBP metabolism after multiple doses in rats. These changes in metabolism were not observed in dogs (there were no statistically significant differences in Cₘₐₓ and AUC₀₋ₜ between the first and last administration of BZP and BNBP).

BZP was higher in the lung tissue within 50 minutes of administration, and the fat content was the highest at 110 minutes of administration. The concentration of BZP in the brain is relatively low, and it is eliminated quickly, and it cannot be detected after 50 minutes. BZP is rapidly converted to BNBP in vivo (not via NADPH coenzyme/CYP450). Among them, liver tissue has the strongest ability to transform BZP in vitro, and spleen has the lowest ability to transform BZP. BNBP was higher in kidney, liver, and lung tissues when administered for 1 minute. BNBP-OH is the main metabolite of BNBP (via NADPH coenzyme/CYP450). However, the study results showed that the cumulative excretion of BZP, BNBP and BNBP-OH was only 3.12% of the administered dose of BZP, suggesting that BNBP-OH may undergo further metabolism in the body.

### Example 4: Toxicology Study in Animals

In the acute toxicity test, a single intravenous injection of BZP was given to rats and dogs, and the MTDs were 200 mg/kg and 300 mg/kg. The toxic reactions of BZP in the nervous system and respiratory system can be recovered within 15 minutes to 4 hours.

In the long-term toxicity test of BZP intravenously injected once a day in rats for 13 consecutive weeks, all rats in the 150 mg/kg group showed shortness of breath, prone position, occasional salivation, and disappearance of righting reflex at the end of each administration. These gradually returned to normal within 1 hour. Some rats occasionally had soft/loose stools, and no obvious abnormalities were found in hematology, blood biochemistry, eye examination, and urine routine. In the long-term toxicity test of BZP intravenously injected once a day in dogs for 13 consecutive weeks, all dogs in the 150 mg/kg group successively vomited white foam-like substances, salivation, head and whole body tremors, spasms/convulsions, shortness of breath may be seen occasionally, and loose stools may be seen occasionally in some dogs during each administration. 150 mg/kg can still cause mild anemia and liver damage (hepatic weight and coefficient increase, liver cell hypertrophy). In some dogs in the 50 mg/kg group, tremors in the head and limbs, loose stools, vomiting, and salivation may be seen during and after administration. The above symptoms all recovered after 28 days of drug withdrawal. The long-term toxicity test of rats and dogs showed that the NOAEL dose of BZP was 50 mg/kg. The nervous system and respiratory system reactions in the long-term toxicity test were transient reactions, and the slight anemia and damage to the liver also recovered within the observation period.

### Example 5: Phase I Clinical Study of a Single Dose (BZP1601 Trial) and Phase I Clinical Study of Multiple Doses (BZP1702 Trial)

Two Phase I clinical trials of BZP have been completed to investigate its pharmacokinetics of a single dose (BZP1601 trial) and multiple doses (BZP1702 trial) on 48 and 29 healthy subjects, respectively. After a single or multiple intravenous injections of BZP, the AUC₀₋ₜ and Cₘₐₓ of BZP in plasma are linearly related to the dose; however, the linear relationship between the AUC₀₋ₜ and Cₘₐₓ of BNBP and the dose has not yet been determined. There was no significant difference in the pharmacokinetic parameters of BZP and BNBP between subjects of different genders, and there was no obvious drug accumulation after multiple intravenous infusions. It did not find other meaningful metabolites except BNBP in two human trials.

Efficacy and Safety of the Test Drug

Two Phase I clinical trials of BZP have been completed to explore the safety and tolerability of a single dose (BZP1601 trial) and multiple doses (BZP1702 trial) on 50 and 30 healthy subjects, respectively. A single intravenous infusion of BZP in the range of 25-400 mg or multiple intravenous infusions of BZP in the range of 50 to 200 mg has good safety and tolerance, and the incidence of AE does not increase with the dose increase.

TABLE 1
List of clinical studies of Brozopentyl Sodium (BZP)

| | | | Dosage (number of | |
|---|---|---|---|---|
| Study number | | | persons exposed to each treatment) | |
| Status (start | | | Administration route and dosage form | |
| date/completion | | Time of | Brozopentyl | |
| date) | Design/Target | treatment | Sodium (BZP) | Placebo |

Completed study

| | | | | |
|---|---|---|---|---|
| BZP1601 | Phase I, | Single | 12.5 mg/100 mL (2) | 0.9% NaCl (12) |
| Completed (from | randomized, | | 25 mg/100 mL (8) | Intravenous |
| Feb. 27, 2017 | single-blind, | | 50 mg/100 mL (8) | infusion |
| to Feb. 26, 2018) | placebo-controlled, | | 100 mg/100 mL (8) | Total number |
| | dose-escalating, | | 200 mg/100 mL (8) | of exposed |
| | single-dose, safety, | | 300 mg/250 mL (8) | persons: 12 |
| | tolerability and | | 400 mg/250 mL (8) | Number of |
| | pharmacokinetic | | Intravenous infusion | permanent |
| | characteristics in | | Total number of | dropping out: 0 |
| | healthy volunteers | | exposed persons: 50 | |
| | | | Number of | |
| | | | permanent dropping | |
| | | | out: 0 | |
| BZP1702 | Phase I, | Multiple; once | 50 mg (10) | 0.9% NaCl (6) |
| Completed (from | randomized, | on each of the | 100 mg (10) | Intravenous |
| Oct. 9, 2017 | single-blind, | first day and the | 150 mg (10) | infusion |
| to Jul. 13, 2018) | placebo-controlled, | seventh day, and | Intravenous infusion | Total number |
| | dose escalating, | twice on each of | Total number of | of exposed |
| | repeated dose, | the second day | exposed persons: 30 | persons: 6 |
| | safety, tolerability | to the sixth day | Number of | Number of |
| | and pharmacokinetic | (a total of 12 | permanent dropping | permanent |
| | characteristics in | times) | out: 1 (50 mg | dropping out: 0 |
| | healthy volunteers | | group) | |

This form is as of Jun. 21, 2019

Human Pharmacokinetics and Product Metabolism

TABLE 2
List of clinical studies of Brozopentyl Sodium (BZP)

| Agreement | | | | |
|---|---|---|---|---|
| number | | | | |
| Status (start | | | Number of persons | |
| date/completion | Design | Method of | Patient distribution | |
| date) | Target | administration | Baseline characteristics | Main findings |
| BZP1601 | Phase I, | A single | 2 persons in | The geometric means |
| Completed (from | randomized, | intravenous | BZP pre-test group | of T_{1/2} of BZP and BNBP |
| 27 Feb., 2017 | single-blind, | infusion | 8 persons in | (the major metabolite) |
| to 26 Feb., 2018) | placebo-controlled, | | each dose group | after one administration |
| | dose-escalating, | | of BZP | were approximately 8 |
| | safety, tolerability | | 12 persons in | hours and 15 hours. |
| | and pharmacokinetic | | the placebo group | The blood drug level of |
| | characteristics of a | | 58.33% of male | BZP and BNBP reached |
| | single intravenous | | 41.67% of female | the peak blood drug level |
| | infusion of BZP in | | 98.3% of Han | after a single intravenous |
| | healthy volunteers | | nationality | infusion of BZP within |
| | | | 1.7% of other | the dose range of 25-400 mg. |
| | | | ethnicities | The AUC₀₋ₜ and Cₘₐₓ of |
| | | | 30.96 of age | BZP in plasma have a |
| | | | (20.05-53.16 | linear relationship with |
| | | | years old) | the dose; however, the |
| | | | 62.49 kg of | linear relationship |
| | | | weight (SD | between the AUC₀₋ₜ and |
| | | | 7.05) kg | Cₘₐₓ of BNBP and the |
| | | | (50.50-76.50 kg) | dose has not yet been |
| | | | 22.23 kg/m² of | determined. |
| | | | BMI (SD 1.58) | There was no significant |
| | | | kg/m² (19.30 | difference in AUC₀₋ₜ and |
| | | | 25.00 kg/m²) | Cₘₐₓ of BZP and BNBP |
| | | | | between subjects of |
| | | | | different genders. |
| | | | | The AUC₀₋ₜ exposure |
| | | | | ratio of BNBP and BZP |
| | | | | was between 3.06% and |
| | | | | 4.35% after a single |
| | | | | intravenous infusion; the |
| | | | | cumulative excretion rate |
| | | | | of urine in each dose |
| | | | | group was basically the |
| | | | | same. |
| | | | | The total cumulative |
| | | | | excretion rate of BZP |
| | | | | and its metabolite BNBP |
| | | | | through urine and feces |
| | | | | in the 200 mg group was |
| | | | | 0.5%. No significant |
| | | | | metabolites other than |
| | | | | BNBP were found. |
| BZP1702 | Phase I, | Multiple | 10 persons in | The geometric means of |
| Completed | randomized, | intravenous | each dose | T_{1/2} of BZP and BNBP |
| (from Oct.9, 2017 | single-blind, | infusions | group of BZP | after one administration |
| to Jul. 13, 2018) | placebo-controlled, | | 6 persons in | were 5.34-5.57 hours and |
| | dose-escalating, | | the placebo group | 7.78-10.3 hours. The T_{1/2} |
| | safety, tolerability | | 66.67% of male | of BZP and BNBP on the |
| | and pharmacokinetic | | 33.33% of female | 7th day of administration |
| | characteristics of | | 94.44% of Han | were 11.1-13.3 hours and |
| | multiple intravenous | | nationality | 24.8-25.9 hours. |
| | infusions of BZP | | 5.56% of other | The blood drug level of |
| | in healthy | | ethnicities | BZP and BNBP reached |
| | volunteers | | 34.14 of age | steady state on the fifth |
| | | | (SD 7.41) | day after multiple |
| | | | (21.74-48.84 | intravenous infusions of |
| | | | years old) | BZP within the range of |
| | | | 62.62 kg of weight | 50-200 mg. |
| | | | (SD 6.45) kg | The AUC₀₋ₜ and Cₘₐₓ of |
| | | | (53.00-75.00 kg) | BZP in plasma have a |
| | | | 22.10 kg/m² of | linear relationship with |
| | | | BMI (SD 1.65) kg/m² | the dose; however, the |
| | | | (19.20-24.90 kg/m²) | linear relationship |
| | | | | between AUC₀₋ₜ and Cₘₐₓ |
| | | | | of BNBP and dose has |
| | | | | not yet been determined. |
| | | | | There was no significant |
| | | | | accumulation of BZP and |
| | | | | BNBP after multiple |
| | | | | intravenous infusions for |
| | | | | 7 days. There was no |
| | | | | significant difference in |
| | | | | AUC₀₋ₜ and Cₘₐₓ of BZP |
| | | | | and BNBP between |
| | | | | subjects of different |
| | | | | genders. |
| | | | | No significant |
| | | | | metabolites other than |
| | | | | BNBP were found. |

This form is as of Jun. 21, 2019

Abbreviations: SD: Standard Deviation

Pharmacokinetics and Metabolism

BZP1601 trial

Analysis population: Any subjects who have received BZP and undergone biological sample collections and can undergo pharmacokinetic analysis will be included in the pharmacokinetic analysis (excluding the 12.5 mg of pre-study group). The study analysis included 6 BPZ groups (respectively 25 mg, 50 mg, 100 mg, 200 mg, 300 mg, and 400 mg) and the placebo group, and the number of subjects enrolled in each group was 8, 8, 8, 8, 8, 8, 12 subjects, a total of 60 subjects. All 60 subjects enrolled in the formal study were included in the pharmacokinetic analysis set.

Analysis results: After a single dose of BZP in healthy subjects, the geometric means of T_{1/2} of BZP and the metabolite BNBP were approximately 8 hours and 15 hours; the median values of Tₘₐₓ in each dose group were similar, suggesting that BZP and BNBP basically reached the peak blood drug level after the end of administration. The cumulative urine excretion rate of each dose group was basically the same. After a single intravenous infusion of different doses of BZP, the main exposure parameters AUC₀₋ₜ and Cₘₐₓ of BZP in plasma are linearly related to the dose within the range of 25-400 mg; the linear relationship between AUC₀₋ₜ and Cₘₐₓ of BNBP and the dose cannot be conclusively concluded. In the dose range of 25-400 mg, the main PK exposure parameters AUC₀₋ₜ and Cₘₐₓ of BZP and BNBP were not significantly different between subjects of different genders. In the 200 mg group, the cumulative excretion rate of BZP and its metabolite BNBP through urine and feces was 0.5%, and no debrominated metabolites were found in the metabolites.

BZP1702 Trial

Analysis population: same as BZP1601 trial. The study contained 4 BZP groups (50 mg, 100 mg, 200 mg respectively) and a placebo group. Each group enrolled 10, 10, 10 and 6 subjects, respectively, a total of 36 subjects. 1 subject in the 50 mg group took a drug two days before the administration, and the paracetamol, caffeine, artificial cow-bezoar and chlorphenamine maleate may affect the metabolism of the drug, and the subject dropped out of the group after collecting a 24-hour blood sample on the second day of administration, so the pharmacokinetic parameters could not be fully calculated, and thus this subject was not included in the pharmacokinetic analysis data set, and the other 29 subjects who were given BZP were all included in the pharmacokinetic analysis data set.

Analysis results: within the dose range of 50 to 200 mg, BZP and BNBP have basically reached a steady state on the 5th day after continuous multiple intravenous infusion of BZP. After the first administration and steady-state administration, the AUC₀₋ₜ and Cₘₐₓ of BZP have a linear relationship with the dose, but the linear relationship between the AUC₀₋ₜ and Cₘₐₓ of BNBP and the dose cannot be determined. After 7 days of continuous administration, the accumulation ratio of BZP was 1.10-1.18, and that of BNBP was 1.60-1.78. The main pharmacokinetic exposure parameters (AUC₀₋ₜ and Cₘₐₓ) of BZP and BNBP were not significantly different between subjects of different genders (the mean ratio ranges of BZP were 0.797-1.01 and 0.799-1.01, and the mean ratio ranges of BNBP were 0.845-1.20 and 0.851-1.12). No significant metabolites other than BNBP were found.

Safety and Tolerability

Number of Exposed Patients and Duration of Exposure

BZP1601 Trial

Analysis population: Any subjects who received study drug and had at least one safety data collection will be included in the safety analysis (excluding the 12.5 mg of pre-study group). All 60 subjects enrolled in the formal study were included in the safety data set, with 48 subjects in the BZP group and 12 subjects in the placebo group.

All subjects completed intravenous infusion according to the requirements of the regimen. The total dose of BZP is shown in the table below.

TABLE 3
Total dose of Brozopentyl Sodium (BZP)

| Items | 25 mg | 50 mg | 100 mg | 200 mg | 300 mg | 400 mg | Placebo |
|---|---|---|---|---|---|---|---|
| Number of subjects | 8 | 8 | 8 | 8 | 8 | 8 | 12 |
| enrolled | | | | | | | |
| Mean (mg) (standard | 25 (0) | 50 (0) | 100 (0) | 200 (0) | 300 (0) | 400 (0) | 0 (0) |
| deviation) | | | | | | | |

Data source: BZP1601 Clinical Trial Report Form 12-1

BZP1702 Trial

Analysis population: same as BZP1601 trial. All 36 subjects enrolled were included in the safety analysis data set, 30 subjects in the BZP group and 6 subjects in the placebo group.

Except for one subject in the 50 mg group who only completed the first administration due to dropping out midway, the other subjects completed 12 administrations at a specific dose according to the requirements of the regimen. The total dose of BZP is shown in the table below.

TABLE 4
Total Dose of Brozopentyl Sodium (BZP)

| Items | 50 mg | | 100 mg | | 200 mg | | Placebo |
|---|---|---|---|---|---|---|---|
| Enrollment | 10 | | 10 | | 10 | | 6 |
| Total dose of | | | | | | | |
| Brozopentyl | | | | | | | |
| Sodium (mg) | | | | | | | |
| Mean (standard | 545.00 | (173.93) | 1200 | (0) | 2400 | (0) | 0 (0) |
| deviation) | | | | | | | |
| Min, max | 50, 600 | | 1200, 1200 | | 2400, 2400 | | 0, 0 |
| Total times of | | | | | | | |
| administrations | | | | | | | |
| (times) | | | | | | | |
| Mean (standard | 10.9 | (3.48) | 12 | (0) | 12 | (0) | 12 (0) |
| deviation) | | | | | | | |

| | | | | |
|---|---|---|---|---|
| Min, max | 1, 12 | 12, 12 | 12, 12 | 12, 12 |

Data source: BZP1702 Clinical Trial Report Form 12-1

Summary of Phase I Clinical Trial:

Two Phase I clinical trials of BZP have been completed. The AUC₀₋ₜ and Cₘₐₓ of BZP in plasma are linearly related to the dose after a single intravenous infusion of BZP in the dose range of 25-400 mg (BZP1601 trial) or multiple intravenous infusions in the dose range of 50-200 mg (BZP1702 trial). However, the linear relationship between AUC₀₋ₜ and Cₘₐₓ of BNBP and the dose has not yet been determined. There was no significant drug accumulation after multiple intravenous infusions. Only the T_{1/2} geometric means of BZP and BNBP observed in the two trials were different. In the BZP1601 trial, after administration of BZP once, T_{1/2} geometric means of BZP and BNBP was about 8 hours and 15 hours; while in the BZP1702 trial, they were 5.34-5.57 hours and 7.78-10.3 hours. It is speculated that the difference between the two trials may be related to the time of blood sample collection. The blood samples were collected at 0 minutes, 20 minutes, 40 minutes, minutes (end of infusion), 1.25 hours, 1.5 hours, 2 hours, 2.5 hours, 3 hours, 4 hours, 6 hours, 8 hours, 12 hours, 16 hours, 24 hours, 36 hours, and 48 hours after administration in BZP1601 trial, while in the BZP1702 trial, on the first day of administration, the blood samples were collected only by 24 hours after administration.

BZP was shown to be safe and well tolerated in healthy subjects. In the two clinical trials, only one case of moderate hypotension occurred in the multiple dose trial (100 mg group), and the AE of others was mild. There was no statistical difference in the proportion of AE between the BZP groups and the placebo group, and the incidence rate did not increase with dose increase. AE judged by the investigator to be related to study drug included mild bradycardia (a single dose of 25 mg and 200 mg without any clinically significant ECG QTc abnormalities), decreased white blood cell counts (a single dose of 50 mg), decreased neutrophil cell count/percentage (a single dose of 50 mg, multiple doses of placebo), first-degree atrioventricular block (a single dose of 100 mg), proteinuria (a single dose of 200 mg), coagulopathy (a single dose of 200 mg), administration site pain (multiple doses of 200 mg), hypotension (multiple doses of 100 mg), hypokalemia (multiple doses of 200 mg) and decreased diastolic blood pressure (multiple doses of 200 mg). The AE related to the study drug occurred from a few minutes to the 9th day after administration, and the symptoms turned to disappear without any clinical intervention. There was no death, SAE, AE leading to drop out, or other important AE in both clinical trials, nor were there pregnancy reports. No dose-limiting toxicity (DLT) occurred.

### Example 6: Phase II Clinical Trial

The BZP1703 clinical trial is a multi-center, randomized, double-blind, placebo-controlled Phase II clinical trial. The purpose of the trial is to evaluate the safety and feasibility of different doses of BZP for injection compared with placebo in the treatment of acute ischemic stroke, and to preliminarily explore its effectiveness, so as to provide a basis for the design of Phase III clinical trial.

The trial plans to enroll 375 acute ischemic stroke patients aged 45 to 75 in China. The subjects participating in the trial will be randomly assigned to the following three treatment groups according to the ratio of 2:2:1: BZP low-dose test group (100 mg bid, intravenous infusion, each administration of 2 vials of BZP and 2 vials of BZP simulation agent), BZP high-dose test group (200 mg bid, intravenous infusion, each administration of 4 vials of BZP), placebo control group (bid, intravenous infusion, each administration of 4 vials of BZP simulation agent). The subjects will receive the treatment of test drug twice a day for 14 consecutive days, and then enter the follow-up period. The subjects will be followed up for safety on the 45th and 90th days, and carry out the evaluation of NIHSS and the modified Rankin scale (mRS). Each subject is expected to participate in the trial for approximately 91 days.

The primary safety endpoint of the BZP1703 trial is SAE, and the primary efficacy endpoint is the difference between the NIHSS score on day 14 and the baseline NIHSS score. Secondary safety endpoints include all-cause death, fatal bleeding and symptomatic intracranial hemorrhage, hemorrhagic transformation of cerebral infarction detected by head magnetic resonance imaging (MRI) on day 14, various adverse events; other bleeding events, AE, AEs of special interest (seizures, bradycardia, atrioventricular block, coagulation abnormalities, proteinuria, leukopenia), number and incidence of patients with new vascular events (ischemic stroke/hemorrhagic stroke/myocardial infarction/vascular death), vital signs, physical examination, laboratory tests (blood routine, blood biochemistry, urine routine, stool routine, coagulation function, etc.), 12-lead electrocardiogram (ECG) and other abnormalities. Secondary efficacy endpoints include change from baseline in NIHSS score at days 7 and 90, rate of reduction from baseline in NIHSS score at days 7, 14, and 90, and proportion of subjects with NIHSS score 0-1 at days 7, 14, and 90 or decrease of ≥4 scores, incidence of neurological worsening NIHSS score increase of ≥4 scores at days 7 and 14, ordinal analysis of distribution of mRS scores at days 7, 14, and 90, proportion of subjects with mRS scores of 0-1, 0-2, and 0-3 on days 7, 14 and 90, the incidence of new cerebral infarction shown on head MRI-DWI on day 14, etc.

1.1 Design Features

The BZP1703 trial is a multi-center, randomized, double-blind, placebo-controlled clinical trial. The subjects participating in the trial will be randomly assigned into the following three treatment groups according to the ratio of 2:2:1: BZP low-dose test group (100 mg bid), BZP high-dose test group (200 mg bid), placebo control group (bid).

The trial used a central randomization system to randomize participants to reduce potential bias. After confirming that the subject meets all the requirements for participating in the trial, the investigator will randomize the subject through the interactive web response system before the first administration of the test drug. Subjects will be randomly assigned to three groups A/B/C with a unique random number in a 2:2:1 ratio. The researchers, the sponsor and the subjects were not aware of the corresponding relationship between the three groups A/B/C and the low-dose test group, high-dose test group and placebo control group. The specific groups of A/B/C are not known until the unblinding.

The trial will blind the subjects, investigators, other researchers in the research center and sponsor (including monitors and contract research organizations) to ensure that there is no bias in the analysis and evaluation of safety and efficacy. In order to ensure blindness, the packaging form and administration method of the simulation agent lyophilized powder injection used in the dosage regimen are the same as those of the test drug. When all data is entered into the database and confirmed to be correct, the database will be locked according to the process. Once the database is locked, the unblinding process can be initiated. According to the process, the randomized independent statistician released the subject random table to the project statistician for statistical analysis of the data after unblinding.

1.2 Results and Discussion

Preliminary results from this trial showed that a single dose of 200 mg was significantly more effective than a single dose of 100 mg; the dosage regimen of a single dose of 200 mg twice daily was significantly more effective than the dosage regimen of a single dose of 100 mg twice daily.

1. A method for preventing or treating cardio-cerebral ischemic diseases or improving cardio-cerebral circulation disorders or anti-thrombosis in an individual, the method comprising administering an effective amount of sodium 5-bromo-2-(α-hydroxypentyl)benzoate (BZP) compound, wherein the BZP compound has the following structure:

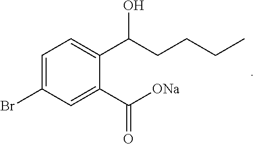

2. The method according to claim 1, wherein the daily dose of BZP is 50-500 mg, such as 100-500 mg, 200-500 mg, 250-500 mg, 300-500 mg, 325-475 mg, 350-475 mg, 350-450 mg, 375-450 mg, or 375-425 mg, for example 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg or 500 mg.

3. The method according to claim 1, wherein the single dose of BZP is 25-400 mg, such as 50-400 mg, preferably 50-300 mg, more preferably 50-250 mg, even more preferably 100-250 mg, such as 25 mg, 50 mg, 75 mg, 100 mg, 125 mg, 150 mg, 175 mg, 200 mg, 225 mg, 250 mg, 275 mg, 300 mg, 325 mg, 350 mg, 375 mg or 400 mg; and/or the BZP is administered three times a day, twice a day, once a day, once every two days, once every three days, twice a week, once a week, once every two weeks, or once every four weeks.

4. The method according to claim 1, wherein BZP is administered once to three times a day, with a dose of 50-250 mg each time, for example, once, twice or three times a day, with a dose of 50 mg, 75 mg, 100 mg, 125 mg, 150 mg, 175 mg, 200 mg, 225 mg or 250 mg each time.

5. The method according to claim 1, wherein BZP is administered twice a day, with a dose of 100-250 mg each time, such as twice a day, with a dose of 100 mg, 125 mg, 150 mg, 175 mg, 200 mg, 225 mg or 250 mg each time, preferably twice a day, with a dose of 200 mg each time.

6. The method according to any one of claims 1-5, wherein the disease is acute stroke and related diseases, preferably, the disease is mild or moderate acute ischemic stroke.

7. The method according to claim 6, wherein the disease is a disease characterized by:
(1) Acute ischemic stroke;
(2) Acute ischemic stroke, cerebral anterior circulation infarction;
(3) Acute ischemic stroke, cerebral anterior circulation infarction, non-cardiogenic infarction;
(4) Acute ischemic stroke, cerebral anterior circulation infarction, non-cardiogenic infarction, moderate severity (NIHSS 5-15 scores);
(5) Acute ischemic stroke, cerebral anterior circulation infarction, non-cardiogenic infarction, moderate severity (NIHSS 5-15 scores), aged 45-85 years; or
(6) Acute ischemic stroke, anterior cerebral circulation infarction, non-cardiogenic infarction, moderate severity (NIHSS 5-15 scores), aged 45-85 years, BZP needs to be administered within 24 hours of stroke onset.

8. A method according to any one of the preceding claims, wherein the individual is a human.

9. The method according to any one of the preceding claims, wherein the individual has a time from stroke onset to drug administration of not more than 24 hours, preferably the individual is a human patient with a time from stroke onset to drug administration of 6-24 hours.

10. A method according to any one of the preceding claims, wherein the treatment achieves one or more of the following improved effects in the individual:
(1) Improving the neurological function score and reducing the NIHSS score;
(2) Improving neurological function outcomes and reducing mRS scores;
(3) Reducing the volume of cerebral infarction;
(4) Reducing the recurrence of ischemic stroke;
(5) Reducing the hemorrhagic transformation of ischemic stroke, including symptomatic intracranial hemorrhage and asymptomatic hemorrhagic transformation; and/or
(6) Reducing the mortality of ischemic stroke.

11. The method according to any one of the preceding claims, wherein the dosage regimen has a lower incidence of adverse events than other dosage regimens and achieves better improved effects than other dosage regimens.

12. The method according to any one of the preceding claims, wherein BZP is administered by parenteral route, preferably by intravenous injection or intravenous infusion.

13. The method according to any one of the preceding claims, wherein BZP is administered in the form of a pharmaceutical composition such as sterile lyophilized powder or an injection reconstituted from sterile lyophilized powder in a pharmaceutically acceptable liquid carrier.

14. A use of BZP according to claim 1 in the manufacture of a medicament for preventing or treating cardio-cerebral ischemic diseases or improving cardio-cerebral circulation disorders or anti-thrombosis in individuals.

15. The use according to claim 14, wherein the daily dose of BZP is 50-500 mg, such as 100-500 mg, 200-500 mg, 250-500 mg, 300-500 mg, 325-475 mg, 350-475 mg, 350-450 mg, 375-450 mg, or 375-425 mg, for example 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg or 500 mg.

16. The use according to claim 14, wherein the single dose of BZP is 25-400 mg, such as 50-400 mg, preferably 50-300 mg, more preferably 50-250 mg, even more preferably 100-250 mg, such as 25 mg, 50 mg, 75 mg, 100 mg, 125 mg, 150 mg, 175 mg, 200 mg, 225 mg, 250 mg, 275 mg, 300 mg, 325 mg, 350 mg, 375 mg or 400 mg; and/or the BZP is administered three times a day, twice a day, once a day, once every two days, once every three days, twice a week, once a week, once every two weeks, or once every four weeks.

17. The use according to claim 14, wherein BZP is administered once to three times a day, with a dose of 50-250 mg each time, for example, once, twice or three times a day, with a dose of 50 mg, 75 mg, 100 mg, 125 mg, 150 mg, 175 mg, 200 mg, 225 mg or 250 mg each time.

18. The use according to claim 14, wherein BZP is administered twice a day, with a dose of 100-250 mg each time, such as twice a day, with a dose of 100 mg, 125 mg, 150 mg, 175 mg, 200 mg, 225 mg or 250 mg each time, preferably twice a day, with a dose of 200 mg each time.

19. The use according to any one of claims 14-18, wherein the disease is acute stroke and related diseases, preferably, the disease is mild or moderate acute ischemic stroke.

20. The use according to any one of claims 14-19, wherein the drug is in the form of a sterile lyophilized powder or an injection reconstituted from sterile lyophilized powder in a pharmaceutically acceptable liquid carrier.

21. A single pharmaceutical dosage unit, characterized by comprising sodium 5-bromo-2-(α-hydroxypentyl)benzoate (BZP) compound, wherein the single pharmaceutical dosage unit comprises the BZP compound in the dose of 25-400 mg, such as 50-400 mg, preferably 50-300 mg, more preferably 50-250 mg, even more preferably 100-250 mg, for example 50 mg, 75 mg, 100 mg, 125 mg, 150 mg, 175 mg, 200 mg, 225 mg, 250 mg, 275 mg, 300 mg, 325 mg, 350 mg, 375 mg or 400 mg.

22. The single pharmaceutical dosage unit according to claim 21, wherein the BZP compound is in the form of a sterile lyophilized powder or an injection reconstituted from sterile lyophilized powder in a pharmaceutically acceptable liquid carrier.

23. The single pharmaceutical dosage unit according to claim 21 or 22, wherein the pharmaceutically acceptable liquid carrier is sterile water, Ringer's solution, phosphate-buffered saline, or isotonic sodium chloride solution.

24. The single pharmaceutical dosage unit according to any one of claims 21-23, which is in the form of a vial for injection, an ampoule, an infusion bag, a prefilled needle or a prefilled syringe, a solution or sterile lyophilized powder containing the BZP compound according to claim 22.

25. A pharmaceutical kit, comprising one or more single pharmaceutical dosage units according to any one of claims 21-24, and optionally a package insert for instructing the use of the single pharmaceutical dosage units.
